(19) United States
(12) Patent Application Publication
(10) Pub. No.: US 2011/0300242 A1
(43) Pub. Date: Dec. 8, 2011

(54) Novel botanical formulation for treating sickle cell disease

(76) Inventors: Swami Nathan, Piscataway, NJ (US); Robert Swift, Fort Collins, CO (US)

(21) Appl. No.: 12/802,437

(22) Filed: Jun. 8, 2010

(51) Int. Cl. A61K 36/67; A61P 7/00; A61P 7/06

(52) U.S. Cl. 424/734

(57) ABSTRACT

An improved plant medicament composition, comprising a mixture of extractions of Piper guineense, Eugenia caryophyllum and Sorghum bicolor plant material, without the use of Pterocarpus osun stem, for treating sickle cell disease is disclosed. A method for the preparation of said composition is also provided.

### FIELD OF THE INVENTION

The present invention relates to the field of botanical formulations, particularly it relates to phytodrug or plant medicament for treatment and management of sickle cell disease and methods of preparing and using same.

### BACKGROUND OF INVENTION

Sickle cell disease (SCD) is a hereditary blood disorder, affecting over 75,000 people in the United States. Sickle cell disease (SCD) affects over twenty million people throughout the world and is particularly common among those whose ancestors come from sub-Saharan Africa, Spanish-speaking regions in the Western Hemisphere (South America, the Caribbean, and Central America), Saudi Arabia, India, and Mediterranean countries such as Turkey, Greece, and Italy.

In the U.S., those with SCD have an average mortality in their 40s, a poor quality of life and high medical costs. In SCD, a mutation in β-globin causes deoxygenated sickle hemoglobin (deoxy-HbS) to form insoluble polymers inside red blood cells (RBCs), which deforms the RBCs into rigid shapes or sickle cells that occlude capillaries and small blood vessels. The only disease-modifying drug approved for use in SCD patients is hydroxyurea, an anti-cancer drug. Not all patients respond to hydroxyurea, and it can be poorly tolerated causing myelosuppression in some patients. Despite extensive studies on SCD by researchers over several decades, there has been little progress in the development of additional disease modifying agents. Therefore, new, safer and more effective therapeutic anti-sickling agents are needed to treat patients with SCD, particularly children, which could improve the quality of life, increase the life expectancies, and reduce the estimated 100,000 hospitalizations and $500 million in direct hospital costs due to sickle cell disease in the United States. Sickle cell disease occurs in 1 in every 500 African American births,
and 1 in 12 African Americans has sickle cell trait. The morbidity and mortality factors associated with sickle cell disease are well-known and the acute and chronic trauma of the painful episodes is indescribable. In view of these realities, there is a desperate need for drugs or agents that could alleviate the effects of this terrible disease, and the search for such agents is of the highest priority.

U.S. Pat. No. 5,800,819 describes a composition requiring extracts from at least four plant materials, viz., Piper guineense, Eugenia caryophyllata, Sorghum bicolor and Pterocarpus osun for treating sickle cell disease. The present invention provides a distinctly different and improved plant formulation that is more efficacious against SCD and simpler to formulate.

### SUMMARY OF INVENTION

It is an object of this invention to provide an improved and efficacious botanical medicament for the treatment and management of sickle cell disease.

It is an additional object of this invention to provide a method for the preparation of an improved plant formulation for treating SCD.

Various other objects and advantages of the invention will become obvious from the detailed description of the invention.

### DETAILED DESCRIPTION OF THE INVENTION

Various objects and advantages of the invention are achieved by preparing a botanical composition, comprising a mixture of extractions of Piper guineense, Eugenia caryophyllum and Sorghum bicolor plant material, without the use or extraction of Pterocarpus osun stem, said mixture containing a drug material effective for treating sickle cell disease. The composition may further comprise pharmaceutically acceptable carrier, excipients, buffers, emollients, and the like, well known to one of ordinary skill in the art to which this invention belongs.
A typical procedure for extraction as an example is now set forth.

Sorghum bicolor seeds were obtained from USDA (ID #IS2724, REDHAGARI). The seeds were grown under normal green house conditions. Canadian pot mixture fortified with anhydrous ferric chloride (10 mg /pot) was used as the growth medium. Watering was done with tap water once a week. Every month the leaves were removed and air dried. The dried leaves were extracted with aqueous sodium bicarbonate (7.4% concentration W/V at a pH ranging from about 8 to about 8.3) by stirring for about 8 hrs at normal ambient temperature. The extract was allowed to settle, then decanted, filtered, centrifuged and freeze dried.

Piper guineense seeds and Eugenia caryophyllum fruit were extracted similarly. The freeze dried components were thoroughly blended to form a uniform mixture, which could be formulated as tablets, granules, solutions, capsules and the like by well known methods. The sickle cell bioassay was carried out by using blood removed from a sickle cell subject and exposed to the extract in vitro to measure antisickling activity employing counting and imaging techniques as described in Iyamu, et al, British Journal of Hematology, (2002), 118: 337-343.

### COMPARATIVE RESULTS OF BIOASSAY

| | | Drug | Bioassay |
|---|---|---|---|
| | Solvent used for | Concentration | (% of cells |
| Sample Description | Extraction | (mg/ml) | still sickled) |
| Control | | 0 | 95 |
| Sorghum bicolor, | Sodium bicarbonate | 5 | 62 |
| Piper guineense | (pH 8-8.3) | | |
| and Eugenia | | | |
| caryophyllum | | | |
| Sorghum bicolor, | Sodium bicarbonate | 10 | 46 |
| Piper guineense | (pH 8-8.3) | | |
| and Eugenia | | | |
| caryophyllum | | | |
| Sorghum bicolor, | Sodium bicarbonate | 15 | 26 |
| Piper guineense | (pH 8-8.3) | | |
| and Eugenia | | | |
| caryophyllum | | | |
| Sorghum bicolor, | Sodium bicarbonate | 20 | 8 |
| Piper guineense | (pH 8-8.3) | | |
| and Eugenia | | | |
| caryophyllum | | | |
| Nicosan | | 5 | 88 |
| U.S. Pat. No. | | | |
| 5,800,819 | | | |
| Nicosan | | 10 | 67 |
| U.S. Pat. No. | | | |
| 5,800,819 | | | |
| Nicosan | | 15 | 42 |
| U.S. Pat. No. | | | |
| 5,800,819 | | | |
| Nicosan | | 20 | 18 |
| U.S. Pat. No. | | | |
| 5,800,819 | | | |

These results indicate that a formulation comprising a mixture of extracts obtained from Sorghum bicolor, Piper guineense, and Eugenia caryophyllum plant material, but devoid of Pterocarpus osun extract, is a potent agent having as much as two-fold greater anti-sickling efficacy compared to the prior art formulation disclosed in U.S. Pat. No. 5,800,819 when prepared under similar condition, but with a lower preferred pH of 8.3.

1. A botanical composition, comprising a mixture of extractions from Piper guineense, Eugenia caryophyllum, and Sorghum bicolor plant material, without the use of Pterocarpus osun stem, said mixture containing a drug material effective for treating sickle cell disease.

2. The composition of claim 1 further comprising any pharmaceutically acceptable carrier and excipients.

3. The composition of claim 2, wherein said extraction is formulated as a solution, suspension, powder, tablet or capsule.

4. A method for preparing a botanical composition, comprising the steps of:
obtaining an extract having anti-sickling activity from Piper guineense, Eugenia caryophyllum and Sorghum bicolor plant material; and
(ii) preparing said extract in a suitable form for treating sickle cells.

5. The method of claim 4, comprising the steps of:
(i) extracting Piper guineense seeds, Eugenia caryophyllum fruit and Sorghum bicolor leaves with a 7.4% weight by volume aqueous solution of sodium bicarbonate at a pH ranging from about 8.0 to about 8.3 by stirring for about 8hr at normal ambient temperature;
(ii) allowing the extract to settle, then decanting, filtering, centrifuging and freeze drying the resulting material; and
(iii) thoroughly blending the freeze dried material to form a uniform mixture.

6. The method of claim 5, administering said blended uniform mixture for treating sickle cell disease, in a tablet, capsule, solution or granular form.
